(19) United States
(12) Patent Application Publication
(10) Pub. No.: US 2026/0198967 A1
(43) Pub. Date: Jul. 16, 2026

(54) APPARATUS AND METHOD FOR IMAGE-GUIDED COLPOPEXY

(71) Applicant: Kyongtae BAE, Gangnam-gu, Seoul (KR)

(72) Inventors: Kyongtae BAE, Gangnam-gu, Seoul (KR); Junu BAE, Henderson, NV (US); Sonu BAE, Henderson, NV (US); Choonkyu KIM, Deogyang-gu, Goyang-si, Gyeonggi-do (KR)

(21) Appl. No.: 19/134,852

(22) PCT Filed: Dec. 4, 2023
(86) PCT No.: PCT/KR2023/019757

§ 371 Date: Jun. 2, 2025

Related U.S. Application Data
(60) Provisional application No. 63/385,829, filed on Dec. 2, 2022.

(51) Int. Cl. A61B 17/34; A61B 17/00; A61B 17/42; A61B 34/00; A61B 34/20

(52) U.S. Cl. CPC A61B 17/3468; A61B 34/20; A61B 34/71; A61B 2017/00004; A61B 2017/3405; A61B 2017/4225

(57) ABSTRACT

Devices and methods are disclosed for performing image-guided colpopexy, such as sacrospinous ligament colpopexy, to treat pelvic organ prolapse. The devices are configured for transvaginal or subcutaneous access approach, comprising rotatable and translatable vaginal device with image-guiding markers, anchoring device, retention anchors, and pulley and pusher wires. By precise placement of a vaginal device and an anchoring device, a path between the vaginal wall and pelvic ligament is established and used to insert a retention assembly that enables juxtaposition and fastening of the vaginal wall to the pelvic ligament for colpopexy.

### TECHNICAL FIELD

This invention relates to a treatment of pelvic organ prolapse by image-guided fixation of the vaginal wall to pelvic ligament. Medical imaging allows the display and visualization of a patient's internal pelvic structures. The vagina and pelvic ligaments are identified and accessed with medical interventional devices under the guidance of imaging without surgical incisions. By precise placement of a vaginal device and an anchoring device, a path between the vaginal wall and pelvic ligament is established and used to insert a retention assembly that enables juxtaposition and fastening of the vaginal wall to the pelvic ligament for colpopexy. The image-guided colpopexy devices are configured for transvaginal or subcutaneous access approach.

### BACKGROUND ART

Almost 50% of women older than 50 years of age suffer from pelvic organ prolapse (POP). Although many women with POP do not require specific treatment, some of them experience impaired quality of life including negative sense of body image, sexual dysfunction, urinary dysfunction, and bowel dysfunction that require clinical management.

There are multiple options in management of POP. Non-invasive methods include biofeedback therapy, pelvic floor muscle strengthening exercises, or intravaginal support devices (e.g., pessaries). When non-invasive treatment options fail to manage symptoms, invasive treatment such as surgery is considered.

Surgery for POP can be classified as either obliterative or reconstructive surgery. Obliterative surgery involves narrowing, shortening, or completely closing the vagina to provide additional support to organs that prolapse through the vagina or press against the walls of the vagina. Reconstructive surgery aims to support the pelvic floor and retain the prolapsing organs to their original position. The various reconstructive surgical options available to patients can be categorized by the surgical approach (e.g., abdominal, vaginal, laparoscopic, or robotic approach), the type of repair (e.g., native tissue, mesh, or graft repair), and any concomitant procedures (e.g., hysterectomy).

Abdominal reconstructive surgery for POP commonly involves sacrocolpopexy or sacrohysteropexy, where the vaginal apex or uterus is posteriorly anchored to the sacral promontory using surgical mesh. Vaginal reconstructive surgery performed without use of synthetic mesh includes anterior and posterior colporrhaphy, uterosacral ligament suspension, sacrospinous ligament fixation, and iliococcygeus fascia suspension procedures. Uterosacral ligament suspension is performed via an in-traperitoneal approach, whereas sacrospinous ligament fixation and iliococcygeus fascia suspension are performed via an extraperitoneal route. The traditional uterosacral ligament suspension involves plicating the uterosacral ligament at or above the ischial spine level, while a modified uterosacral ligament suspension uses a passage of the suture through the coccygeus-sacrospinous ligament muscle complex. Compared to sacrospinous ligament fixation, iliococcygeus fascia suspension uses the fascia of the iliococcygeus muscle below the ischial spine and lateral to the rectum as the suture site for attaching the vaginal vault. Posterior intravaginal slingplasty or infracoccygeal sacropexy is a typical vaginal reconstructive surgery performed with use of synthetic mesh.

Although surgery is highly effective in relieving POP symptoms in 85% to 100% of patients, it constitutes a major financial burden to the healthcare system. With the annual incidence of POP surgery ranging from 1.5 to 1.8 cases per 1,000 woman-years, approximately 11% of women undergo POP surgery by the age of 80 years. Furthermore, up to 30% of women who undergo surgery require a re-operation, adding to the total cost of POP surgery of over 1 billion dollars per year in the United States alone. In addition, postoperative complications including neurologic symptoms, ureteral obstruction, and urinary tract infection occur in 15%-30% of patients. In particular, use of transvaginal meshes has been associated with high risks of mesh erosion, pain, and immobility. In April 2019, the FDA ordered the cessation of selling and distributing surgical mesh products for transvaginal repair of POP.

As patients with POP are often elderly and have limited tolerance for extensive surgical procedures, it is highly desirable to implement a safe, non-invasive, and efficient treatment method without requiring a prolonged and risky surgical operation. Therefore, it is crucial to develop a therapeutic method that is highly effective in the treatment of POP and is relatively easily implantable using a minimally invasive interventional procedure.

Our image-guided colpopexy system is based on the consideration that the target vaginal vault and pelvic ligament for colpopexy intervention can be precisely identified and accessed under guidance of medical imaging. The vaginal colpopexy device is introduced to the target anatomical structures via a transvaginal or subcutaneous route to perform colpopexy without a large incision or major disturbance of surrounding tissues. The potential target pelvic ligaments include uterosacral ligament, sacrospinous ligament, and iliococcygeus fascia, depending on the indication of clinical applications and accessibility of the device.

### DISCLOSURE OF INVENTION

### Solution to Problem

In order to perform an image-guided colpopexy, the patient is imaged to identify and localize the vagina wall and pelvic ligaments that are the target anatomies for intervention. While the vaginal wall is readily approachable by directly placing a vaginal probe into the vagina, the target pelvic ligament deep inside the pelvis is accessed via a transvaginal or subcutaneous route.

The transvaginal approach of image-guided colpopexy begins with placement of a TV (transvaginal) vaginal device over the pelvis of the patient who is lying in the supine position on the imaging scanner table. The TV vaginal device consists of a pelvic flap, a control hub, and a TV vaginal probe. The pelvic flap is placed and fastened over the pelvis of the patient to secure and stabilize the control hub for the colpopexy procedure. The TV vaginal probe is inserted through the center of the control hub into the vaginal lumen. The patient is imaged to identify the TV vaginal probe and target pelvic ligaments. The TV vaginal probe contains a needle guide track. The distal tip of the TV vaginal probe near the vaginal vault is visualized and confirmed on imaging. The distal tip of the TV vaginal probe has an opening side-port for the needle guide track through which a needle is projected into a target pelvic ligament.

Under image guidance, the TV vaginal probe is shifted and rotated to nudge the vaginal vault toward the target pelvic ligament and align the needle guide track to the target pelvic ligament. The orientation of the TV vaginal probe is achieved by rotations about two different axes: swinging the control hub about the y-axis (i.e., the axis directing from anus to pubis in the supine position) while keeping the pelvic flap as the fixed reference; and rotating the TV vaginal probe about the horizontal x-axis (i.e., the long axis of the vaginal lumen) by dialing the control hub clockwise or counter-clockwise centered at the vaginal lumen.

The path from the distal tip of the needle guide track to the target pelvic ligament is estimated to provide the distance and rotation angle that are required for a needle to reach from the distal tip of the needle guide track to the target pelvic ligament. The distance and rotation angle are more precisely computed by the needle navigation program or manually from the analysis of the 3D images of the pelvis containing the TV vaginal device.

Following the appropriate placement and rotation of the needle guide track and confirmation of its trajectory toward the target pelvic ligament, an anchoring device is introduced through the needle guide track. The anchoring device consists of a needle, head, body, handle, and retainer control gears. The hollow lumen of the needle is loaded prior to or during the intervention with a retainer that is pushed forward by turning the knobs of the retainer control gears. The needle of the anchoring device is inserted through the needle guidance track of the TV vaginal probe and gently advanced beyond the distal opening of the needle guidance track. The needle penetrates the vaginal wall and traverses the pelvic extraperitoneal space fat into the target pelvic ligament. The manipulation of the retainer control gears releases the distal anchor of the retainer from the needle to the target pelvic ligament.

With the confirmation of the release of the distal anchor of the retainer, the retainer and distal pulley wire are gently pulled back. As a consequence of pulling, the distal anchor splays and apposes to the target ligament. With the withdrawal of the needle to the vaginal lumen under image guidance, the proximal anchor of the retainer is released and placed at the vaginal lumen. The pulley wires, anchoring device and TV vaginal device are removed. At the completion of the transvaginal colpopexy procedure, the vaginal wall apposed by the proximal anchor of the retainer is fastened to the target ligament apposed by the distal anchor via the retention wire, i.e., forming a colpopexy.

The subcutaneous approach of image-guided colpopexy begins with insertion of a SC (subcutaneous) vaginal probe into the vaginal lumen. The patient is placed in the prone or semi-decubitus position on the imaging scanner table to uncover and direct the buttocks for the placement of the needle of the anchoring device. The patient is imaged to identify the SC vaginal probe and target pelvic ligaments. The distal tip of the SC vaginal probe near the vaginal vault is visualized and confirmed by the imaging system. The distal tip of the SC vaginal probe has an opening side-port that is the final target for the needle insertion of the anchoring device. Under image guidance, the path from a suitable subcutaneous skin site via the target pelvic ligament to the vaginal port is estimated to provide the distance and angle that are required for a needle to navigate. The distance and rotation angle are more precisely computed by the needle navigation program or manually from the analysis of the 3D images of the pelvis containing the SC vaginal device.

An anchoring device, which is of the same function and structure as the anchoring device for the transvaginal approach except for having more straight and stiff needles, is used for the subcutaneous approach. The needle of the anchoring device is inserted from a suitable subcutaneous skin site via the target pelvic ligament to the opening side-port of the vaginal probe following the predetermined path traversing subcutaneous fat, pelvic muscle, target ligament, extraperitoneal fat, and vaginal wall.

After the needle is confirmed with its distal tip within the SC vaginal probe, the manipulation of the retainer control gears releases the distal anchor of the retainer from the needle to the vaginal lumen.

With the confirmation of the release of the distal anchor of the retainer, the retainer and distal pulley wire are gently pulled back. As a consequence of pulling, the distal anchor splays and apposes to the vaginal lumen. With the withdrawal of the needle to the target ligament under image guidance, the proximal anchor of the retainer is released and placed at the target ligament. The pulley wires, anchoring device and SC vaginal device are removed. At the completion of the subcutaneous colpopexy procedure, the vaginal wall apposed by the distal anchor of the retainer is fastened to the target ligament apposed by the proximal anchor via the retention wire, i.e., forming a colpopexy.

The colpopexy described in the present embodiment is performed via a series of operations including placing vaginal probes, imaging, positioning, needle penetration, pushing, pulling, and anchoring, without a large incision or major disturbance of surrounding tissues. Although the sacrospinous ligament is commonly used to anchor the vaginal vault, other pelvic ligament structures such as the uterosacral ligament can be applied for colpopexy. Various medical imaging systems including CT, MRI, ultrasound, fluoroscopy, and laparoscopy can be used for the image guidance. Positioning of the vaginal probes, needles, retainers, and wires can be performed manually, via a motorized mechanism, by operating a hand-operated device.

During or after the colpopexy procedure, it is informative to track and monitor the location and configuration state of the devices and retainers in the body. For this purpose, fiducial markers made of radio-opaque materials including barium sulfate or metal can be detected under fluoroscopy or CT. Some devices may be partially or entirely coated with barium sulfate. Magnetically sensitive fiducial markers can be used to guide procedures in MR imaging.

A first aspect of the invention is A vaginal device for image-guided colpopexy comprising:
at least one pelvic flap to be fastened over a pelvis of a patient to secure and stabilize the vaginal device;
control hub at a center of the vaginal device, the control hub including a rotatable inner tube to be placed in the vaginal lumen, joints rotatable about at least two different axes while keeping the at least one pelvic flap as the fixed reference, and reference marks denoting degree or rotations; and
at least one elongated, curved vaginal probe received through the control hub, the vaginal probe including a proximal opening in the proximal end, a distal opening port in a distal end, a curved needle guide track configured to angulate a needle in predetermined degrees while advancing from the vagina to a target ligament.

In certain embodiments, a vaginal device further comprising locking screws to secure and stabilize the vaginal probe and the control hub.

In certain embodiments, the needle guide track of a vaginal device is curved in the range of 60-80 degrees to angulate the needle while advancing from the vagina to a target ligament.

In certain embodiments, the control hub of a vaginal device is configurated to accomplish advancing and rotating the vaginal probe by rotational and/or translational manipulation thereof, thereby gaining access to a vaginal lumen target site, and aiming at the target ligament as guided by medical imaging.

In certain embodiments, the vaginal probe and/or needle guide track of a vaginal device are curved and angled toward a target pelvic ligament to facilitate angulation of the needle in the predetermined degrees, when the needle is inserted and pushed through the probes.

A second aspect of the invention is an anchoring device for image-guided colpopexy comprising:
at least one needle, curved or straight, with a hollow lumen;
at least one retainer, the retainer including a distal anchor, a proximal anchor, and retention wires joining the distal and proximal anchors;
one or more pulley wires which are connected to any one or more of the proximal anchor and the distal anchor;
a pusher wire which pushes the at least one retainer forward to slide through the lumen of the needle; and
a body to which the needle is connected.

In certain embodiments, wherein the body of an anchoring device comprises at least one head to which the needle is locked or assembled together as one unit.

In certain embodiments, wherein the body of an anchoring device comprises any one or more of control gears, a side knob, a rear knob, a slit ruler for pusher wire, a slit ruler for one or more pulley wires, a sliding indicator and a handle.

In certain embodiments, each of the distal anchor and proximal anchor of an anchoring device is in form of bar, cylinder and foldable umbrellas that are insertable through a hollow lumen, and freely rotatable and swingable at the joint connected to the retention wires when released out of the hollow lumen.

In certain embodiments, the body of an anchoring device comprises a slit ruler for one or more pulley wires and a sliding indicator, and
one or more pulley wires comprise a proximal pulley wire which is connected to the proximal anchor and a distal pulley wire which is connected to the distal anchor,
wherein a proximal extension of the distal pulley wire is placed in the slit ruler for one or more pulley wires, and locked and secured by the sliding indicator.

In certain embodiments, the body of an anchoring device further comprises control gears, a side knob, a rear knob and a slit ruler for one or more pulley wires,
wherein a proximal extension of the pusher wire is placed in between the control gears, threaded through the rear knob, and becomes visible through the slit ruler.

In certain embodiments, a vaginal device further comprises a fiducial marker for tracking the location or position of any one or more components of the device with medical imaging.

In certain embodiments, an anchoring device further comprises a fiducial marker for tracking the location or position of any one or more components of the device with medical imaging.

In certain embodiments, a vaginal device is in part composed of metal, silicon, or biocompatible polymers including biodegradable or non-biodegradable polymers.

In certain embodiments, an anchoring device is in part composed of metal, silicon, or biocompatible polymers including biodegradable or non-biodegradable polymers.

A third aspect of the invention is

An apparatus for image-guided colpopexy comprises a vaginal device and an anchoring device,
wherein the vaginal device comprising:
at least one pelvic flap to be fastened over a pelvis of a patient to secure and stabilize the vaginal device;
control hub at a center of the vaginal device, the control hub including a rotatable inner tube to be placed in the vaginal lumen, joints rotatable about at least two different axes while keeping the at least one pelvic flap as the fixed reference, and reference marks denoting degree or rotations; and
at least one elongated, curved vaginal probe received through the control hub, the vaginal probe including a proximal opening in the proximal end, a distal opening port in a distal end, a curved needle guide track configured to angulate a needle of the anchoring device in predetermined degrees while advancing from the vagina to a target ligament,
wherein the anchoring device comprising:
at least one needle, curved or straight, with a hollow lumen;
at least one retainer, the retainer including a distal anchor, a proximal anchor, and retention wires joining the distal and proximal anchors;
one or more pulley wires which are connected to any one or more of the proximal anchor and the distal anchor;
a pusher wire which pushes the at least one retainer forward to slide through the lumen of the needle; and
a body to which the needle is connected.

A fourth aspect of the invention, A method for image-guided colpopexy using the vaginal device, said method comprising steps of:
(a) placing the vaginal device in pelvis of a patient;
(b) inserting the vaginal probe into the vaginal lumen with its distal tip close to a vaginal vault;
(c) performing medical imaging of the pelvis with one or more selected from CT, MRI, ultrasound, fluoroscopy, and laparoscopy to display and visualize the vaginal probe in vaginal vault and target pelvic ligament;
(d) indicating locations of the distal opening port of the vaginal probe and the target pelvic ligament on medical imaging;
(e) moving the vaginal probe to nudge the vaginal vault toward the target pelvic ligament and aim the distal opening port of the vaginal probe at the target pelvic ligament; and
(f) obtaining three-dimensional coordinates of the distal opening port of the vaginal probe and the target ligament to determine the path from the distal opening port of the vaginal probe to the target pelvic ligament.

In certain embodiments, the method is for the transvaginal approach, further using the anchoring device, comprising steps of:
(a′) projecting a needle with a hollow lumen to traverse a vaginal wall into a target pelvic ligament;
(b′) placing a retainer, one or more pulley wires, and a pusher wire through the lumen of the needle prior to or during the intervention;
(c′) pushing the retainer by the pusher wire to release only the distal anchor from the needle into the target pelvic ligament;
(d′) withdrawing the needle to place its tip within the vaginal lumen;
(e′) pushing the proximal anchor by the pusher wire to release the proximal anchor from the needle to the vaginal lumen; and
(f′) removing the one or more pulley wires, pusher wire, needle, and vaginal probe.

In certain embodiments, the method for the transvaginal approach further comprising one or more steps of:
confirming the engagement of the distal anchor to the pelvic ligament by using medical imaging or gently pulling the one or more pulley wires; and
confirming the engagement of the proximal anchor to the vaginal wall by using medical imaging or gently pulling the one or more pulley wires.

In certain embodiments, the method is for the subcutaneous approach further using the anchoring device, comprising steps of:
(a″) obtaining three-dimensional coordinates of a subcutaneous skin site from medical imaging to estimate a needle path from the subcutaneous skin site through a target ligament to a distal opening port of a vaginal probe, including the distance and angle of the needle navigation;
(b″) inserting a needle with a hollow lumen from the subcutaneous site to traverse the target ligament, and vaginal wall into the distal opening port of the vaginal probe;
(c″) placing a retainer, one or more pulley wires, and a pusher wire through the lumen of the needle prior to or during the intervention;
(d″) pushing the retainer by the pusher wire to release only the distal anchor of the retainer from the needle into the vaginal lumen;
(e″) withdrawing the needle to place its tip at the target ligament;
(f″) pushing the proximal anchor of the retainer by the pusher wire to release the proximal anchor from the needle to the target ligament; and
(g″) removing the one or more pulley wires, pusher wire, needle, and vaginal probe.

In certain embodiments, the method for the subcutaneous approach further comprising one or more steps of:
confirming the engagement of the distal anchor to the vaginal wall by using medical imaging or gently pulling the one or more pulley wires; and
confirming the engagement of the proximal anchor to the target ligament by using medical imaging or gently pulling the one or more pulley wires.

In certain embodiments, positioning of a vaginal probe, a needle, a retainer, one or more pulley wires, and a pusher wire is performed via a motorized mechanism, performed manually, or performed by operating a hand-operated device.

### BRIEF DESCRIPTION OF DRAWINGS

FIG. 1A-1C are transverse cross-sectional drawings of the female pelvic anatomy in the supine position showing the relative positions of vaginal vault, bilateral sacrospinous ligaments, other internal pelvic organs, and a transvaginal (TV) vaginal probe with a needle placed in the vaginal lumen.

FIG. 2A-2D are 3D rendered and wireframe perspective drawings of a TV vaginal device.

FIG. 3A is an image of a prototype TV vaginal device prior to placement over pelvis.

FIG. 3B-3C are two images of a human subject lying supine on a table with a prototype TV vaginal device placed over pelvis.

FIG. 4A is a schematic perspective drawing of the female pelvic anatomy showing the pelvic bony structure, right sacrospinous ligament, vaginal lumen, vaginal vault, and a vaginal probe within the vaginal lumen.

FIG. 4B is a transverse cross-sectional drawing of the female pelvic anatomy in the supine position showing the vaginal vault, bilateral sacrospinous ligaments, other internal pelvic organs, and the distal tip of a vaginal probe placed near the vaginal vault.

FIG. 5A is the same as FIG. 4A but with a vaginal probe whose distal opening port is shifted and redirected toward a target right sacrospinous ligament.

FIG. 5B is the same as FIG. 4B but with a vaginal probe whose distal opening port is shifted and redirected toward a target right sacrospinous ligament.

FIG. 6A is the same as FIG. 5A but with a vaginal probe that contains a needle projected from the opening port to a right target sacrospinous ligament.

FIG. 6B is the same as FIG. 5B but with a vaginal probe that contains a needle projected from the opening port to a right target sacrospinous ligament.

FIG. 7A is a 3D rendered perspective drawing of a TV vaginal device through which an anchoring device is inserted.

FIG. 7B is a wireframe perspective drawing of a TV vaginal device through which an anchoring device is inserted.

FIG. 7C is an image of a prototype anchoring device for the transvaginal approach.

FIG. 7D is an image of a human subject lying supine on a table with a prototype TV vaginal device through which a prototype anchoring device is inserted for the transvaginal approach.

FIG. 8A is a 3D rendered perspective drawing of an anchoring device for the transvaginal approach.

FIG. 8B is a magnified view of FIG. 8A over the body of the anchoring device.

FIG. 8C is a cross-sectional view of a needle through which a retainer, two pulley wires, and a pusher wire are inserted.

FIG. 8D is a wireframe perspective drawing of an anchoring device for the transvaginal approach following the placement of a distal pulley wire and a pusher wire.

FIG. 8E is the same as FIG. 8B following the placement of a distal pulley wire and a pusher wire.

FIG. 8F is an illustration of the distal and proximal anchors of a retainer apposing to a ligament and a vaginal wall which are fastened by the retention wire connected to the distal and proximal anchors of the retainer.

FIG. 8G is a cross-sectional view of a needle through which a retainer with folded umbrella-type and bar-type anchors, and a proximal pulley wire.

FIG. 8H is the same as FIG. 8F except for one bar-type anchor replaced by an expanded umbrella-type anchor.

FIG. 9A is a CT image of human subject pelvis with a TV vaginal probe containing a needle placed to the right sacrospinous ligament.

FIG. 9B is a CT image of human subject pelvis with the placement of the distal anchor of a retainer at the right sacrospinous ligament and the proximal anchor in the vaginal lumen.

FIG. 9C is a schematic perspective drawing of the female pelvic anatomy showing the vaginal vault fastened to the right sacrospinous ligament by means of a retainer.

FIG. 10 is a 3D rendered perspective drawing of a subcutaneous (SC) vaginal device for the subcutaneous approach.

FIG. 11A is a schematic perspective drawing of the female pelvic anatomy showing the pelvic bony structure, right sacrospinous ligament, vaginal lumen, vaginal vault, and a SC vaginal probe within the vaginal lumen.

FIG. 11B is a transverse cross-sectional drawing of the female pelvic anatomy in the prone position showing the vaginal vault, bilateral sacrospinous ligaments, other internal pelvic organs, and the distal tip of a SC vaginal probe placed near the vaginal vault.

FIG. 12A is the same as FIG. 11A but with a SC vaginal probe whose port is shifted and redirected toward a target right sacrospinous ligament that is traversed by a subcutaneously inserted needle entering into the SC vaginal probe port.

FIG. 12B is the same as FIG. 11B but with a SC vaginal probe whose port is shifted and redirected toward a target right sacrospinous ligament that is traversed by a subcutaneously inserted needle entering into the SC vaginal probe port.

FIG. 13A is a 3D rendered perspective drawing of a SC vaginal device for the subcutaneous approach with a needle inserted into the distal port of the vaginal probe.

FIG. 13B is a magnified drawing of a SC vaginal probe for the subcutaneous approach with an inserted needle through which a retainer anchor and partially exposed retention wire are deployed into the distal port of the vaginal probe.

FIG. 14 is an image of a human subject lying prone on a table with a SC vaginal probe into which a needle is inserted from the right buttock for the subcutaneous approach.

### MODE FOR THE INVENTION

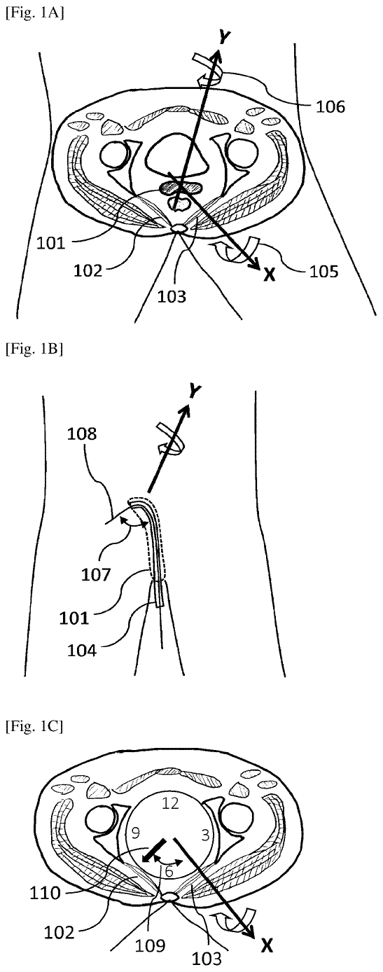

FIG. 1A-1C show the key anatomical structures in the female pelvis that are pertinent to colpopexy: vagina 101, right sacrospinous ligament 102, and left sacrospinous ligament 103. FIG. 1B represents the pelvis in the coronal plane, while FIG. 1C represents the pelvis in the axial plane. Given the location and orientation of sacrospinous ligaments relative to the vagina in 3D anatomy, a vaginal probe 104 requires rotations about two axes (roll-axis and yaw-axis) to aim at a target sacrospinous ligament from the vaginal vault. Following the convention of the aircraft principal axes, the roll rotation 105 corresponds to the rotation about the x-axis while the yaw rotation 106 corresponds to the rotation about the y-axis. The yaw angle 107, which is the angle between the long-axes of the vaginal probe 104 and a needle 108, is in the range of 60-80 degrees as shown in the coronal plane of the pelvis in FIG. 1B. The roll angle 109 of the vaginal probe 104 to aim at a target sacrospinous ligament, to be described in terms of a clock-face representing the cross-section of the vagina, is in the range of 7-8 o'clock (denoted by arrow 110) targeting at the right sacrospinous ligament 102 or in the range of 4-5 o'clock targeting at the left sacrospinous ligament 103, as shown in the axial plane of the pelvis in FIG. 1C.

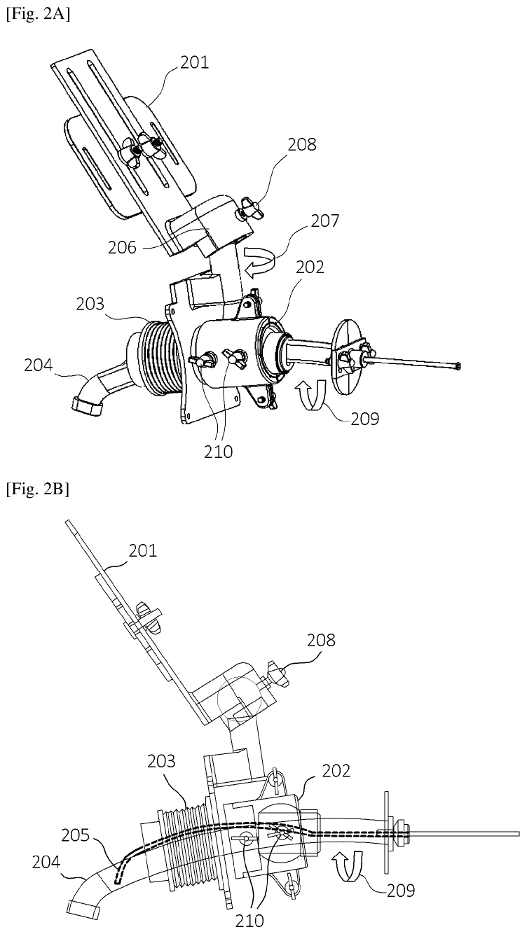

FIG. 2A-2D show a transvaginal (TV) vaginal device which has the pelvic flap 201 to be fastened over the pelvis of the patient to secure and stabilize the control hub 202 for the colpopexy procedure. The inner tube 203 of the control hub 202, which is placed inside of the vaginal lumen, is configured to be smooth and rotate freely within the vaginal lumen. The TV vaginal probe 204 is inserted through the center of the control hub 202 into the vaginal lumen. The TV vaginal probe 204 contains the needle guide track 205. The distal tip of the TV vaginal probe 204 has an opening for the needle guide track 205 through which a needle is projected into a desired direction.

The distal ends of the TV vaginal probe 204 and needle guide track 205 are smoothly curved and angled toward a target pelvic ligament to facilitate the angulating of a needle in the range of 60-80 degrees, when the needle is inserted and pushed through the needle guide track 205 to access the target pelvic ligament.

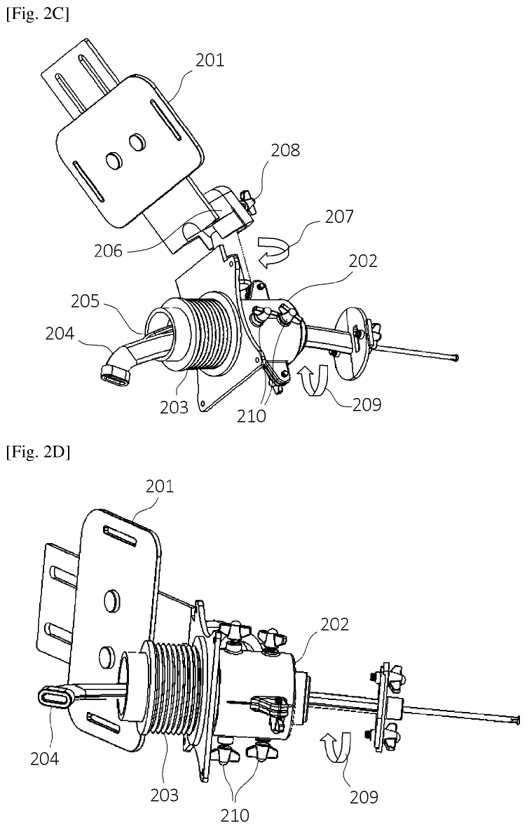

The control hub 202 can rotate at the joint 206 connected to the pelvic flap 201 following the rotation angle indicated by the arrow 207. This rotation allows the TV vaginal probe 204 to rotate about the y-axis (i.e., the axis directing from anus to pubis in the supine position) while keeping the pelvic flap as the fixed reference, as shown in FIG. 1A-1B. Once the position and orientation of the control hub 202 is determined, the control hub is secured at the pelvic flap 201 by locking screws 208. The TV vaginal probe 204 can also rotate centered at the control hub 202 as indicated by the arrow 209. This rotation allows the TV vaginal probe 204 to rotate about the horizontal x-axis (i.e., the long axis of the vaginal lumen) by dialing the control hub 202 clockwise or counter-clockwise centered at the vaginal lumen, as shown in FIGS. 1A and 1C. Once the position and direction of the TV vaginal probe 204 is determined, the probe is secured at the control hub 202 by locking screws 210. An image of a prototype TV vaginal device is shown in FIG. 3A. Two images of a human subject lying supine on a table with a prototype TV vaginal device placed over the pelvis are shown in FIG. 3B-3C. The control hub displays the clock-face reference marks denoting rotation angles of the vaginal probe.

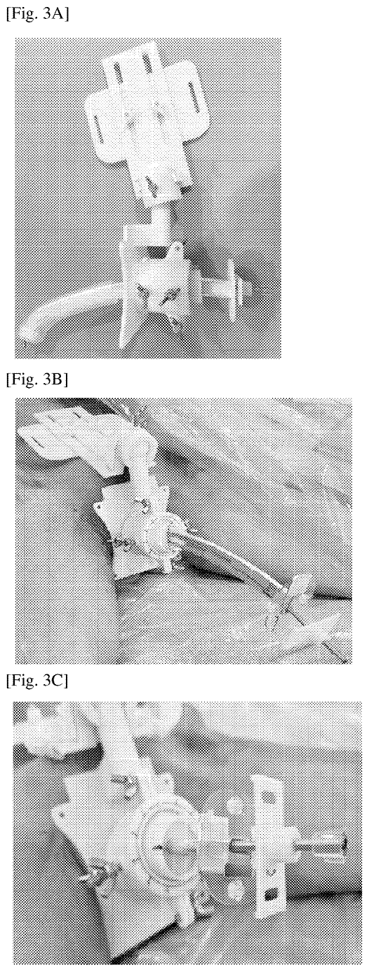

FIGS. 4A and 4B show the placement of a TV vaginal probe 401 in the vaginal lumen 402 for the transvaginal approach for colpopexy. In this approach as depicted in the cross-sectional anatomical view, the patient is in the supine position shown in FIG. 3B. The distal tip 403 of the TV vaginal probe near the vaginal vault 404 is visualized and confirmed on imaging. A target right sacrospinous ligament 405 is identified.

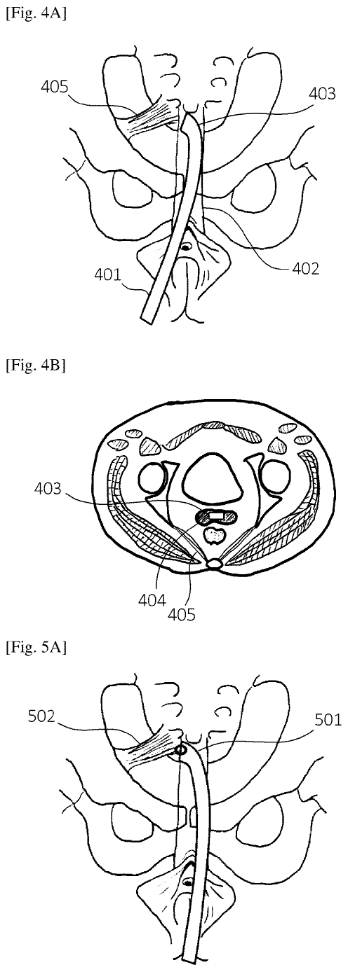

FIGS. 5A and 5B show the distal tip 501 of the TV vaginal probe shifted and rotated to nudge the vaginal vault toward a target right sacrospinous ligament 502 to direct the opening port 503 of the vaginal probe to the target right sacrospinous ligament 502.

This positional manipulation is performed under image guidance. The path from the distal tip of the needle guide track of the vaginal probe to the target pelvic ligament is estimated to provide the distance and rotation angle that are required for a needle to reach from the distal tip of the needle guide track to the target pelvic ligament. The typical distance from the distal tip of the needle guide track at the vaginal wall to a target pelvic ligament is in the range of 4-6 cm. The distance and rotation angle are more precisely computed by the needle navigation program or manually from the analysis of the 3D images of the pelvis containing the TV vaginal device.

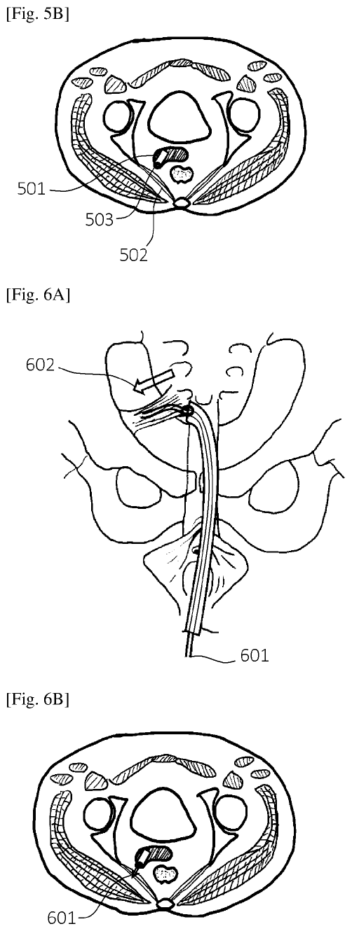

Following the advancement and rotation of the needle guide track 205 of the TV vaginal probe 204 in FIG. 2B and confirmation of its trajectory toward the target pelvic ligament, an anchoring device is introduced through the needle guide track 205. FIGS. 6A and 6B show a needle 601, which is a part of an anchoring device, projected from the opening port of the vaginal probe to penetrate the vaginal wall and traverse the pelvic extraperitoneal space fat into the target pelvic ligament, to the direction indicated by an arrow 602.

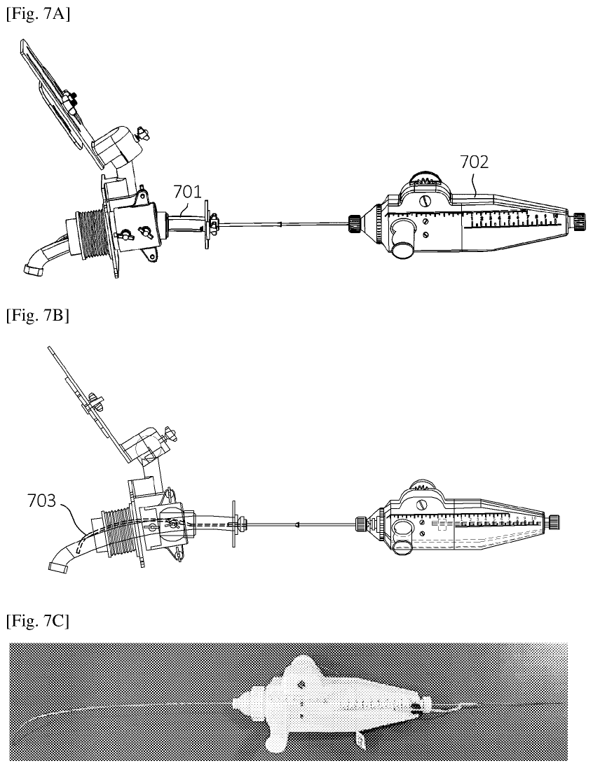

FIG. 7A shows a TV vaginal probe 701 through which an anchoring device 702 is inserted. Specifically, the needle 703 of the anchoring device 702 is inserted through the needle guide track of the vaginal probe 701. The image of a prototype anchoring device is presented in FIG. 7C. A prototype anchoring device inserted to a prototype TV vaginal device placed on a human subject lying supine is shown in FIG. 7D.

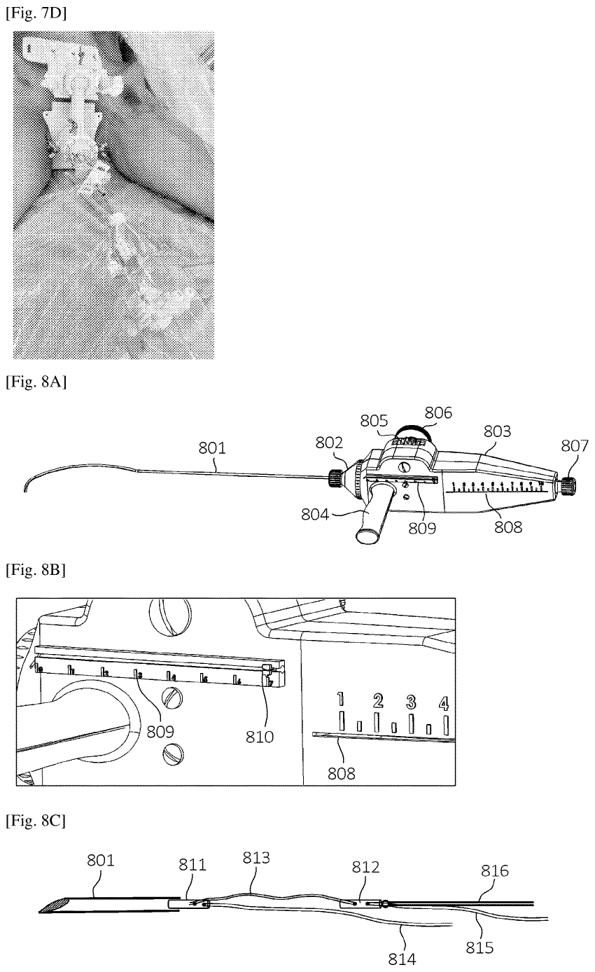

FIG. 8A-B shows an anchoring device which consists of a needle 801, head 802, body 803, and handle 804. The body 803 houses retainer control gears 805 that rotate by turning a side knob 806 or a rear knob 807. The body 803 also contains a slit ruler 808, a slit ruler 809, and a sliding indicator 810. FIG. 8C shows the needle 801 whose hollow lumen is loaded with a retainer which consists of a distal anchor 811, a proximal anchor 812, and a retention wire 813. The lumen of the needle 801 also contains a distal pulley wire 814 which is connected through the body of the distal anchor 811, a proximal pulley wire 815 which is connected through the body of the proximal anchor 812, and a pusher wire 816. The needle including the retainer, pulley wires, and pusher wire is fastened to the header 802. A different type of needle and retainer that are assembled together with a header can be prepared as a unit prior to procedures. Each unit can be exchanged for repeated procedures by locking to the body of the anchoring device. FIG. 8D-E show the proximal extension of the distal pulley wire 814 placed at the slit ruler 809 where the end of the distal pulley wire 814 is locked and secured by the sliding indicator 810. The proximal extension of the pusher wire 816 is placed in between the control gears 805, threaded through the rear knob 807, and becomes visible through the slit ruler 808.

After the placement of the needle 801 of an anchoring device is confirmed with its distal tip positioned at extraperitoneal fat space immediately posterolateral to the target ligament under image guidance, the distal anchor of the retainer 811 is released from the needle by turning the knob 806 or 807 to advance the pusher wire 816. The advancement of the retainer 811-812 is monitored by observing the movement of the pusher wire 816 and distal pulley wire 814 through the slit rulers 808 and 809, respectively, and the migration of the sliding indicator 810.

With the confirmation of the release of the distal anchor 811, the needle 801 is withdrawn under image guidance into the vaginal lumen while the distal pulley wire 814 is pulled back. As a consequence of pulling, the distal anchor 811 splays and apposes to the target ligament 817 in FIG. 8F. After the withdrawal of the needle to the vaginal lumen, further turning forward the knob 806 or 807 advances the pusher wire 815 to release the proximal anchor 812 that splays and apposes to the vaginal wall 818. The proximal pulley wire 815 is removed by pulling only one end of the wire to the direction denoted by arrow 819. The vaginal wall 818 apposed by the proximal anchor 812 is fastened to the target ligament 817 apposed by the distal anchor 811 via the retention wire 813, i.e., forming a colpopexy.

FIG. 8G shows another embodiment of a distal anchor 820, shaped like an umbrella, in a folded configuration within the needle 801. When the distal anchor 820 is uncovered and released from the needle, it expands and apposes to the target ligament 817 shown in FIG. 8H. Apart from the umbrella-shaped distal anchor 820, the design and operation of the remaining parts in FIGS. 8G and 8H are the same as those in FIGS. 8C and 8F, respectively.

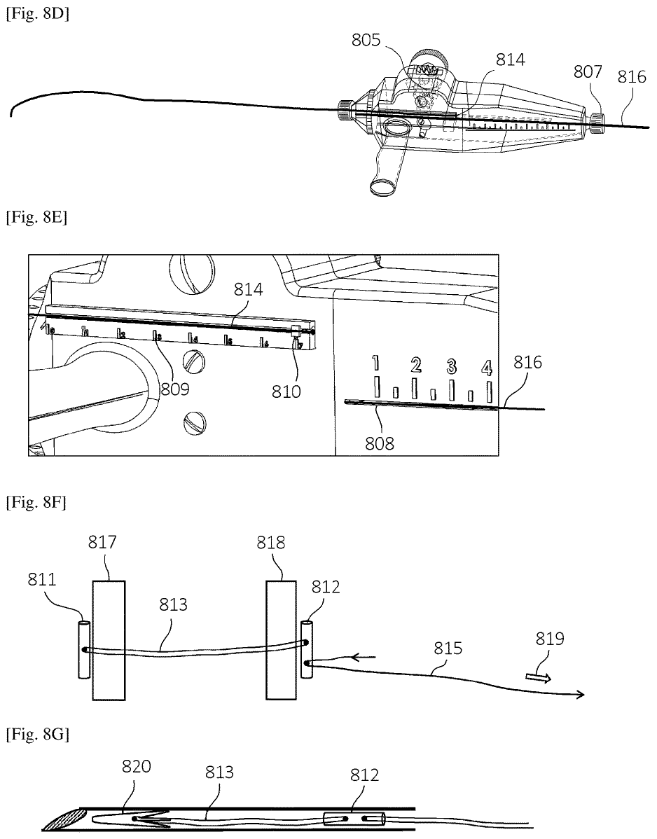

FIG. 9A-B show CT images of human subject pelvis lying supine with a TV vaginal probe 901 placed in the vagina. A metallic needle 902 is inserted through the vaginal probe 901 into the right sacrospinous ligament 903. Linear black lines on CT images are imaging artifacts from metallic objects. A retainer is placed through the needle of an anchoring device with its distal metallic anchor 904 located in the right sacrospinous ligament while the proximal metallic anchor 905 located in the vaginal lumen. At the completion of the transvaginal colpopexy procedure, the vaginal vault apposed by the proximal anchor 905 is fastened to the target ligament apposed by the distal anchor 904, as depicted in FIG. 9C.

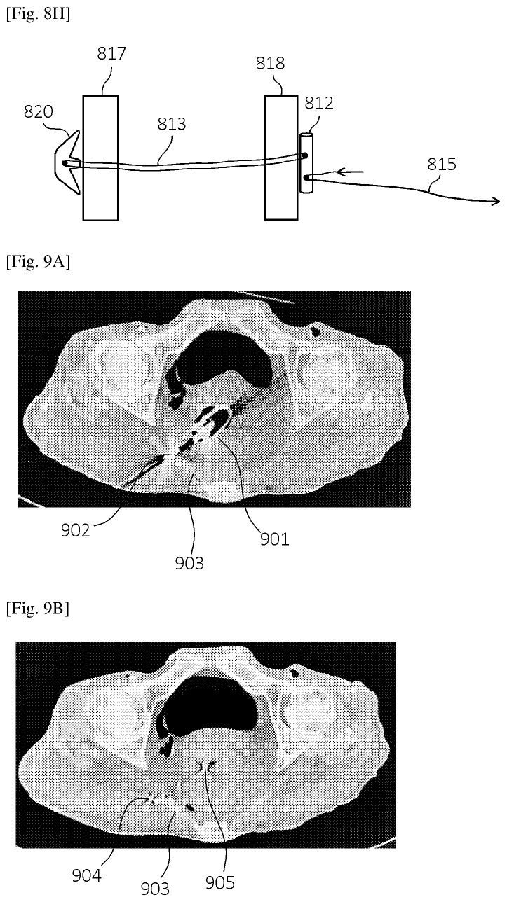

FIG. 10 shows a subcutaneous (SC) vaginal device which has flaps 1001 to be fastened over the pelvis of the patient to secure and stabilize the SC vaginal device for the colpopexy procedure. The center of the SC vaginal device has a control hub 1002 through which 2 cylindrical SC vaginal probes of different calibers, 1003 and 1004, are inserted as one assembled unit. The two SC vaginal probes are open at their proximal ends and have separate opening side ports, 1005 at their distal ends. The two SC vaginal probes are assembled and aligned to have their distal ports coincide. The large-caliber probe 1003 is inserted into the vaginal lumen and advanced to have its distal tip abut or placed near the cervix such that the opening port 1005 of the probe is positioned approximately at the vaginal vault and nudged toward a target pelvic ligament. The large-caliber probe 1003 is also rotated about its long-axis (roll-axis) to have its opening port 1005 directed at the target pelvic ligament. Once the position and direction of the large-caliber probe 1003 is determined, the probe is secured at the control hub 1002 by locking screws 1006. The orientations of the small-caliber probe 1004 can be further modified and fine-tuned by rotating the probe about the yaw-axis to precisely align the opening ports and orientation of the SC vaginal probes from the vagina to a target pelvic ligament. The position and direction of the small-caliber probe 1004 is secured by a locking screw 1007.

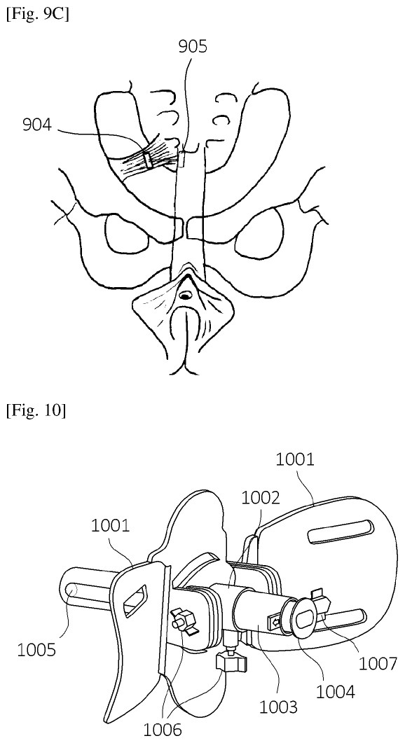

FIGS. 11A and 11B show the placement of a SC vaginal probe 1101 in the vaginal lumen 1102 for the subcutaneous approach for colpopexy. In this approach, the SC vaginal probe 1101 corresponds to the combined large-and small-caliber vaginal probes in FIG. 10. The main function of the SC vaginal probe is to provide its distal opening port 1103 for a conduit for a needle placement from an anchoring device into the vaginal lumen under image guidance. In addition, as depicted in the cross-sectional anatomical view, the patient is in the prone position to uncover and direct the buttocks up for the placement of a needle. Alternatively, the patient may be positioned in a semi-decubitus position to allow for a needle to be placed more vertically toward a target ligament. The distal opening port 1103 of the SC vaginal probe near the vaginal vault 1104 is visualized and confirmed on imaging. A target right sacrospinous ligament 1105 is identified.

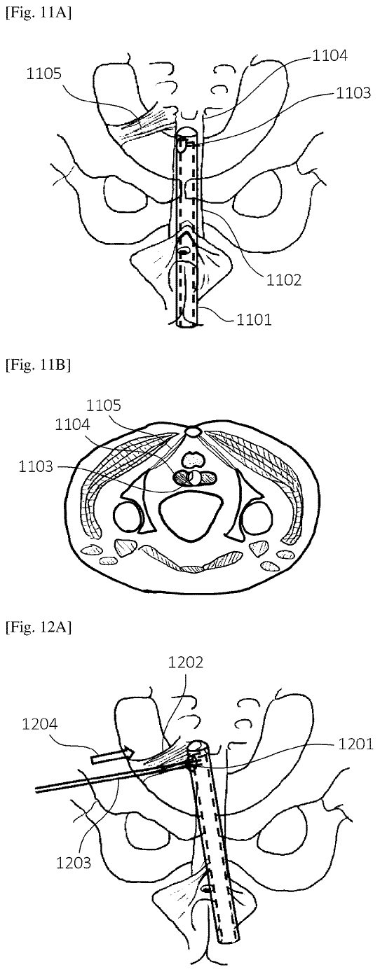

FIGS. 12A and 12B show the distal opening port 1201 of the vaginal probe shifted and rotated to nudge and orient the vaginal vault toward a target right sacrospinous ligament 1202. This positional manipulation is performed under image guidance. The path from a suitable subcutaneous site to the target pelvic ligament 1202 and to the distal opening port 1201 of the vaginal probe is estimated to provide the distance and angle that are required for a needle to navigate. The distance and rotation angle are more precisely computed by the needle navigation program or manually from the analysis of the 3D images of the pelvis containing the SC vaginal device. A needle 1203 from an anchoring device is inserted at the subcutaneous site in the direction denoted by arrow 1204 to traverse subcutaneous fat, pelvic muscle, target ligament, extraperitoneal fat, and vaginal wall into the distal opening port 1201.

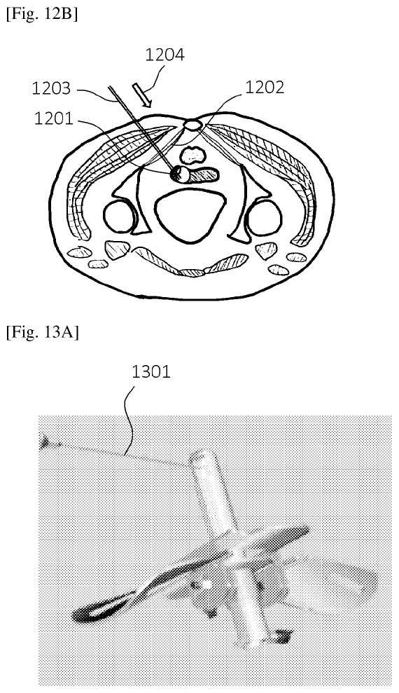

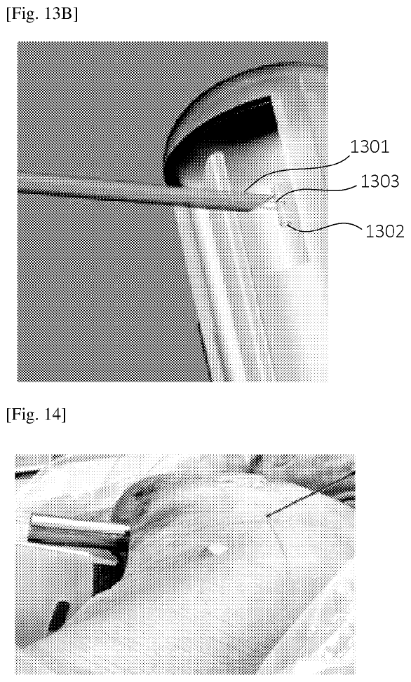

For the subcutaneous approach for colpopexy, the anchoring device used for the transvaginal approach shown in FIG. 8A-H is also used, following the same functionality and operation for anchoring. However, types of needles used between the two approaches may be different. For example, for the subcutaneous approach without using a needle guide track, more straight and stiff needles are preferred, compared to the transvaginal approach in that the trajectory of a needle is prescribed by the needle guide track of the transvaginal probe. FIGS. 13A and 13B show a needle 1301 from an anchoring device inserted into the distal opening port of the vaginal probe. After the needle 1301 is confirmed with its distal tip within the vaginal probe, the distal anchor 1302 of the retainer is released from the needle by turning the knobs of the anchoring device to advance the pusher wire. The advancement of the retainer is monitored through the slit rulers and sliding indicator of the anchoring device. With the confirmation of the release of the distal anchor 1302, the needle 1301 is withdrawn up to extraperitoneal fat space posterolateral to the target ligament while the distal pulley wire is pulled back. Because of pulling, the distal anchor 1302 splays and apposes to the vaginal wall. Further turning forward the knobs of the anchoring device leads to the advancement of the pusher wire to release the proximal anchor into extraperitoneal fat space posterolateral to the target ligament. The proximal anchor is uncovered and released to splay and appose to the target ligament. The pulley wires, vaginal probe, and anchoring device are removed. At the completion of the subcutaneous colpopexy procedure, the vaginal wall apposed by the distal anchor 1302 is fastened to the target ligament apposed by the proximal anchor via the retention wire 1303, i.e., forming a colpopexy, similar to the transvaginal colpopexy shown in FIG. 9C. An image of a human subject lying prone on a table with a SC vaginal probe into which a needle is inserted from the right buttock for the subcutaneous approach, is shown in FIG. 14.

Colpopexy devices including the vaginal probe, anchoring device, and retainer may contain fiducial markers made of radio-opaque materials including barium sulfate or metal to be detected under fluoroscopy or CT. Some devices may be partially or entirely coated with barium sulfate. Magnetically sensitive fiducial markers can be used to guide procedures in MR imaging. Furthermore, parts of the colpopexy devices including the vaginal probe, anchoring device, and retainer are made of metal, biocompatible polymer, or silicon.

1-2. (canceled)

3. An apparatus for image-guided colpopexy comprises a vaginal device and an anchoring device,
wherein the vaginal device comprising:
at least one pelvic flap to be fastened over a pelvis of a patient to secure and stabilize the vaginal device;
control hub at a center of the vaginal device, the control hub including a rotatable inner tube to be placed in the vaginal lumen, joints rotatable about at least two different axes while keeping the at least one pelvic flap as the fixed reference, and reference marks denoting degree or rotations; and
at least one elongated, curved vaginal probe received through the control hub, the vaginal probe including a proximal opening in the proximal end, a distal opening port in a distal end, a curved needle guide track configured to angulate a needle of the anchoring device in predetermined degrees while advancing from the vagina to a target ligament,
wherein the anchoring device comprising:
at least one needle, curved or straight, with a hollow lumen;
at least one retainer configured to be inserted into the hollow lumen of the needle, the retainer including a distal anchor configured to be anchored to a target ligament, a proximal anchor configured to be anchored to a vaginal wall, and retention wires joining the distal and proximal anchors;
one or more pulley wires which are connected to any one or more of the proximal anchor and the distal anchor;
a pusher wire which pushes the at least one retainer forward to slide through the lumen of the needle; and
a body to which the needle is connected.

4. The apparatus for image-guided colpopexy of claim 3, wherein the vaginal device, further comprises locking screws to secure and stabilize the vaginal probe and the control hub.

5. The apparatus for image-guided colpopexy of claim 3, wherein the needle guide track of the vaginal probe is curved in the range of 60-80 degrees to angulate the needle while advancing from the vagina to a target ligament.

6. The apparatus for image-guided colpopexy of claim 3,
wherein the control hub of the vaginal device is configurated to accomplish advancing and rotating the vaginal probe by rotational and/or translational manipulation thereof, thereby gaining access to a vaginal lumen target site, and aiming at the target ligament as guided by medical imaging.

7. The apparatus for image-guided colpopexy of claim 3,
wherein the vaginal probe and/or needle guide track are curved and angled toward a target pelvic ligament to facilitate angulation of the needle in the predetermined degrees, when the needle is inserted and pushed through the probes.

8. The apparatus for image-guided colpopexy of claim 3,
wherein the body of the anchoring device comprises at least one head to which the needle is locked or assembled together as one unit.

9. The apparatus for image-guided colpopexy of claim 3,
wherein the body of the anchoring device comprises any one or more of control gears, a side knob, a rear knob, a slit ruler for pusher wire, a slit ruler for one or more pulley wires, a sliding indicator and a handle.

10. The apparatus for image-guided colpopexy of claim 3,
wherein each of the distal anchor and proximal anchor is in form of bar, cylinder and foldable umbrellas that are insertable through a hollow lumen, and freely rotatable and swingable at the joint connected to the retention wires when released out of the hollow lumen.

11. The apparatus for image-guided colpopexy of claim 3,
wherein the body of the anchoring device comprises a slit ruler for one or more pulley wires and a sliding indicator, and one or more pulley wires comprise a proximal pulley wire which is connected to the proximal anchor and a distal pulley wire which is connected to the distal anchor,
wherein a proximal extension of the distal pulley wire is placed in the slit ruler for one or more pulley wires, and locked and secured by the sliding indicator.

12. The apparatus for image-guided colpopexy of claim 11, wherein the body of the anchoring device further comprises control gears, a side knob, a rear knob and a slit ruler for one or more pulley wires,
wherein a proximal extension of the pusher wire is placed in between the control gears, threaded through the rear knob, and becomes visible through the slit ruler.

13. The apparatus for image-guided colpopexy of claim 3, wherein the vaginal device further comprises a fiducial marker for tracking the location or position of any one or more components of the device with medical imaging.

14. The apparatus for image-guided colpopexy of claim 3, wherein the anchoring device further comprises a fiducial marker for tracking the location or position of any one or more components of the device with medical imaging.

15. The apparatus for image-guided colpopexy of claim 3, wherein the vaginal device is in part composed of metal, silicon, or biocompatible polymers including biodegradable or non-biodegradable polymers.

16. The apparatus for image-guided colpopexy of claim 3, wherein the anchoring device is in part composed of metal, silicon, or biocompatible polymers including biodegradable or non-biodegradable polymers.

17. (canceled)

18. A method for image-guided colpopexy using the apparatus of claim 3, said method comprising steps of:
(a) placing the vaginal device in pelvis of a patient;
(b) inserting the vaginal probe into the vaginal lumen with its distal tip close to a vaginal vault;
(c) performing medical imaging of the pelvis with one or more selected from CT, MRI, ultrasound, fluoroscopy, and laparoscopy to display and visualize the vaginal probe in vaginal vault and target pelvic ligament;
(d) indicating locations of the distal opening port of the vaginal probe and the target pelvic ligament on medical imaging;
(e) moving the vaginal probe to nudge the vaginal vault toward the target pelvic ligament and aim the distal opening port of the vaginal probe at the target pelvic ligament; and
(f) obtaining three-dimensional coordinates of the distal opening port of the vaginal probe and the target ligament to determine the path from the distal opening port of the vaginal probe to the target pelvic ligament,
wherein said method, which is for the transvaginal approach, further using the anchoring device, comprising steps of:
(a′) inserting and projecting the needle of the anchoring device through the needle guide track to traverse a vaginal wall into a target pelvic ligament;
(b′) placing a retainer, one or more pulley wires, and a pusher wire through the lumen of the needle prior to or during the intervention;
(c′) pushing the retainer by the pusher wire to release only the distal anchor from the needle into the target pelvic ligament;
(d′) withdrawing the needle to place its tip within the vaginal lumen;
(e′) pushing the proximal anchor by the pusher wire to release the proximal anchor from the needle to the vaginal lumen; and
(f′) confirming the engagement of the anchors to the pelvic ligament and vaginal wall by using medical imaging or gently pulling the one or more pulley wires; and
(g′) removing the one or more pulley wires, pusher wire, needle, and vaginal probe.

19. (canceled)

20. A method for image-guided colpopexy using the apparatus of claim 3. said method comprising steps of:
(a) placing the vaginal device in pelvis of a patient;
(b) inserting the vaginal probe into the vaginal lumen with its distal tip close to a vaginal vault;
(c) performing medical imaging of the pelvis with one or more selected from CT, MRI, ultrasound, fluoroscopy, and laparoscopy to display and visualize the vaginal probe in vaginal vault and target pelvic ligament;
(d) indicating locations of the distal opening port of the vaginal probe and the target pelvic ligament on medical imaging;
(e) moving the vaginal probe to nudge the vaginal vault toward the target pelvic ligament and aim the distal opening port of the vaginal probe at the target pelvic ligament; and
(f) obtaining three-dimensional coordinates of the distal opening port of the vaginal probe and the target ligament to determine the path from the distal opening port of the vaginal probe to the target pelvic ligament.
wherein said method, which is for the subcutaneous approach further using the anchoring device, comprising steps of:
(a″) obtaining three-dimensional coordinates of a subcutaneous skin site from medical imaging to estimate a needle path from the subcutaneous skin site through a target ligament to a distal opening port of a vaginal probe, including the distance and angle of the needle navigation;
(b″) inserting the needle of the anchoring device from the subcutaneous site to traverse the target ligament, and vaginal wall into the distal opening port of the vaginal probe;
(c″) placing a retainer, one or more pulley wires, and a pusher wire through the lumen of the needle prior to or during the intervention;
(d″) pushing the retainer by the pusher wire to release the first anchor located closer to the needle tip into the vaginal lumen;
(e″) withdrawing the needle to place its tip at the target ligament;
(f″) pushing the second anchor of the retainer by the pusher wire to release the second anchor from the needle to the target ligament;
(g″) confirming the engagement of the anchors to the pelvic ligament and vaginal wall by using medical imaging or gently pulling the one or more pulley wires; and
(h″) removing the one or more pulley wires, pusher wire, needle, and vaginal probe.

21-22. (canceled)
